(19) United States
(12) Patent Application Publication
(10) Pub. No.: US 2026/0219706 A1
(43) Pub. Date: Jul. 30, 2026

(54) Smart Ring Devices

(71) Applicant: Anhui Huami Health Technology Co., Ltd., Hefei (CN)

(72) Inventors: Aijun Zou, Shenzhen (CN); Mingbao Shao, Shenzhen (CN); Tongchun Li, Shenzhen (CN)

(21) Appl. No.: 19/573,334

(22) Filed: Mar. 20, 2026

Related U.S. Application Data
(63) Continuation of application No. 19/395,346, filed on Nov. 20, 2025.
Application No. 19/395,346 is a continuation of application No. PCT/CN2024/120586, filed on Sep. 24, 2024.

(30) Foreign Application Priority Data
Oct. 27, 2023 (CN) 202322922267.3

(51) Int. Cl. G06F 1/16; G06F 1/184; G06F 3/01

(52) U.S. Cl. CPC G06F 1/163; G06F 1/1635; G06F 1/1684; G06F 1/184; G06F 3/011

(57) ABSTRACT

A smart ring device, including a housing, which includes an outer housing, an inner housing and an isolation layer provided between the outer housing and the inner housing, an outer surface of the outer housing defining at least a portion of an outer surface for the smart ring device, an inner surface of the inner housing defining at least a portion of an inner surface for the smart ring device, and an accommodation space being defined at least in part between the isolation layer and at least one of the outer housing or the inner housing; and further including one or more physiological sensors arranged within the accommodation space, wherein the one or more physiological sensors are configured to obtain one or more physiological measurements from a user when the smart ring device is worn by the user.

### CROSS-REFERENCE TO RELATED APPLICATION(S)

The present disclosure is a continuation of United States Application No. 19/395,346, filed November 20, 2025, which is a continuation of International Application No. PCT/CN2024/120586, filed on September 24, 2024, which claims priority and benefit of Chinese Patent Application No. 202322922267.3, filed October 27, 2023, the entire disclosures of all of which are hereby incorporated by reference.

### TECHNICAL FIELD

The present disclosure relates to the technical field of smart wearable devices, and more particularly, to smart ring devices.

### BACKGROUND

With the development of smart wearable technologies, smart ring devices have evolved to incorporate an increasing number of functions. Due to their ergonomic design for finger wear, smart ring devices have emerged as a promising trend for personal health monitoring.

Therefore, how to achieve personal health monitoring using smart ring devices has become a key research focus in the field.

### SUMMARY

The implementations of the present disclosure provides a smart ring device, including: a housing including an outer ring, an inner ring, and an accommodation space between the outer ring and the inner ring; at least one electronic component arranged in the accommodation space; a first electrode and a second electrode, spaced apart circumferentially on the inner surface of the housing and electrically connected to the at least one electronic component; a locating component at least partially arranged within the accommodation space, and a circuit board arranged within the accommodation space.

In some examples, at least a portion of the inner surface of the housing contact finger skin of a user when the smart ring device is worn by the user.

In some examples, a locating structure for securing at least a part of the circuit board includes a first locating component and a second locating component, one of the first locating component or the second locating component is arranged on the circuit board, and the other is arranged on the inner ring or the outer ring.

In some examples, the circuit board includes a locating aperture configured to mate with the locating component for securing the circuit board within the accommodation space.

In some examples, a first end of the locating component is coupled to the outer ring, and a second end of the locating component is coupled to the first electrode or the second electrode via the locating aperture in the circuit board.

In some examples, one end of the locating component is connected to the outer ring, and the other end of the locating component extend through the locating aperture on the circuit board and connect to the first electrode or the second electrode.

In some examples, the circuit board has a shape adapted to the annular circumference of the accommodation space. For instance, the circuit board has a curved surface along the circumference of the accommodation space.

In some examples, the circuit board includes at least two rigid circuit boards and a flexible circuit board connecting the at least two rigid circuit boards, and the at least one electronic component is arranged on the at least two rigid circuit boards.

In some examples, an inner surface of the outer ring includes at least one of a first mounting region and a second mounting region, at least a part of the at least two rigid circuit boards is secured or positioned relative to the first mounting region by a first locating structure, and at least a part of the flexible circuit board is secured to the second mounting region by a second locating structure different from the first locating structure.

In some examples, the rigid circuit board and the flexible circuit board are secured to the housing by same or different locating structures. For instance, the rigid circuit board or the flexible circuit board is secured or positioned relative to the housing by a convex-concave fitting structure, a fastener locating structure, an adhesive locating structure, a magnetic locating structure, a limiting rib/flange structure, or a guiding locating structure or the like.

In some examples, at least one of the rigid circuit board or the flexible circuit board is secured or positioned relative to the housing by cooperation of a locating pin and a mated locating aperture.

In some examples, the first electrode and the second electrode may be arranged on the inner surface of the inner ring and arranged along the circumferential direction of the inner surface of the inner ring. When the smart ring device is worn on a user's finger, the first electrode and the second electrode simultaneously contact the user's finger skin.

In some examples, the first electrode and the second electrode are configured in a shape of semi-annular or quasi-semi-annular. In this way, the first electrode and the second electrode may enclose to form a shape of an closed or generally closed annular configuration, which increases a contact area between the electrodes and the finger skin, thereby facilitating the improvement of detection efficiency and accuracy.

In some examples, at least one of the first electrode or the second electrode extends over more than two-thirds of a width of the inner ring.

In some examples, at least one of the first electrode or the second electrode occupies more than two-thirds of the surface area of the inner surface of the inner ring.

In some examples, the inner ring includes the first electrode, the second electrode and an isolating member between the first electrode and the second electrode along the circumferential direction. The isolating member is made from (e.g., is made of) an electrically insulating material such as a non-metallic material.

In some examples, the inner ring may include an inner support structure, as well as the first electrode and the second electrode arranged on the inner surface of the inner support structure. The inner surface of the inner support structure is configured for contacting the finger skin. The inner support structure may be electrically insulating. The first electrode and the second electrode are spaced apart. The isolating member may include (e.g., is made of) a material same as or different from that of the inner support structure.

In some examples, the outer ring includes an electrically conductive material such as metallic material, and the inner ring includes an electrically insulating material, such as potting material, modable material, polymer, silicone, epoxy, polyester resin or the like.

In some examples, an isolating ring is arranged between the outer ring and the inner ring, and at least a portion of the accommodation space is defined by the isolating ring.

In some examples, the isolating ring includes (e.g., is made of) an electrically insulating material such as a non-metallic material.

In some examples, the isolating ring and the outer ring enclose to define the accommodation space.

In some examples, a first aperture and a second aperture are provided along the circumferential direction of the inner ring, where the first electrode protrudes from the inner surface of the inner ring through the first aperture, and the second electrode protrudes from the inner surface of the inner ring through the second aperture.

In some examples, a first subset of the electronic components in the smart ring device may be disposed on (i.e., secured or otherwise coupled to) one or more rigid circuit boards, and a second subset of the electronic components may be disposed on (i.e., secured or otherwise coupled to) one or more flexible circuit board. Alternatively, substantially all of the electronic components in the smart ring device may be disposed on the one or more rigid circuit boards, and one or more flexible circuit boards are configured to connect adjacent rigid circuit boards. In such cases, the flexible circuit board may be substantially free of electronic components other than interconnection features. In an example, the electronic components included in the smart ring device, other than the connection components, may be all disposed on the one or more rigid circuit boards.

In some examples, an inner surface of the outer ring includes at least one of a first mounting region or a second mounting region. At least a part of the at least two rigid circuit boards is secured or otherwise mounted to the first mounting region, and at least a part of one or more flexible circuit boards is secured or otherwise mounted to the second mounting region.

In some examples, a locating component is arranged on the second mounting region, and a corresponding mating component adapted to engage the locating component is arranged on the flexible circuit board.

In some examples, the rigid circuit board is adhered or otherwise secured to the first mounting region.

In some other examples, at least a portion of the flexible circuit board is adhered or otherwise secured to the second mounting region, while at least a portion of the rigid circuit board is secured to the first mounting region by a locating component arranged on the first mounting region engaging a corresponding mating component arranged on the rigid circuit board.

In some examples, a locating groove is provided on the first or second mounting region, one end of the locating component is fixedly disposed within the locating groove, and the other end of the locating component is connected to the first electrode or the second electrode through the mating component.

In some examples, the locating component includes a locating pin, and the mating component includes a locating aperture.

In some examples, the locating pin may extend through the locating aperture and connect to the first electrode or the second electrode, such as by abutting, gluing, welding or any other connection means.

In an example, the first electrode or the second electrode may include a support portion and an electrode portion. The support portion includes at least two support rods spaced from each other.

In an example, the locating component may extend between adjacent support rods.

In some examples, the at least one electronic component includes a galvanic skin response (GSR) sensing module electrically connected to the first electrode and the second electrode.

In some examples, the at least one electronic component further includes at least one of a body composition sensing module, a charging interface, or a temperature sensing module.

In some examples, the at least one of the body composition sensing module, the charging interface, or the temperature sensing module shares at least one of the first electrode or the second electrode with the galvanic skin response sensing module.

In some examples, the galvanic skin response sensing module is configured to utilize the first electrode and the second electrode to obtain a galvanic skin response signal from a user wearing the smart ring device, and the galvanic skin response signal may be used to analyze an emotional and/or psychological state of the user.

In an example, the smart ring device may further include at least one processing module, configured to process the galvanic skin response signal to obtain a monitoring result of the emotional and/or psychological state of the user. In another example, the smart ring device may further include a wireless communication module, which may transmit the galvanic skin response signal obtained by the galvanic skin response sensing module, either raw data or after one or more types of pre-processing is performed, to another electronic device for processing to obtain the monitoring result.

In some examples, the smart ring device further includes an optical detection module arranged within the accommodation space. The optical detection module includes a plurality of optical components and a plurality of convex lenses positioned in correspondence with the optical components. A plurality of openings are provided on the inner surface of the inner ring, and the plurality of convex lenses protrude from the inner ring through the openings.

In an example, the plurality of convex lenses may form a plurality of convex protrusions, such as racetrack-shaped convex protrusions.

In some examples, the smart ring device further includes an optical detection module arranged within the accommodation space, and the first electrode and the second electrode are symmetrically arranged on opposite sides of the optical detection module.

In some examples, the optical detection module includes at least one light emitter and at least one light detector spaced apart from each other along a circumferential direction of the housing of the smart ring device.

In an example, the optical detection module includes a plurality of light emitters and a light detector, where the plurality of light emitters symmetrically arranged around the light detector along the circumferential direction of the housing. Alternatively, the optical detection module includes a light emitter and a plurality of light detectors, where the plurality of light detectors symmetrically arranged around the light emitter along the circumferential direction of the housing. Alternatively, the optical detection module includes a plurality of light emitters and a plurality of light detectors, where the plurality of light emitters and the plurality of light detectors are arranged in an alternating pattern along the circumferential direction of the housing.

In an example, the at least one light emitter and the at least one light detector form a plurality of optical paths, where a first optical path and a second optical path among the plurality of optical paths have different optical transmission distances.

In some examples, the smart ring device further includes an optical detection module arranged within the accommodation space, where the optical detection module includes a light emitter, as well as a first light detector and a second light detector symmetrically arranged on opposite sides of the light emitter. The light emitter is located in a region where the isolating component connecting the first electrode and the second electrode.

In some examples, the smart ring device further includes a curved battery assembly, where the circuit board and the curved battery assembly are arranged in different regions of the accommodation space along the circumferential direction of the housing.

In some examples, the circuit board and the battery assembly may enclose to form a ring-like or a generally annular shape, and a gap between the circuit board and the curved battery assembly may be offset from a gap between the first electrode and the second electrode. In an example, the gap between the circuit board and the curved battery assembly and the gap between the first electrode and the second electrode may be uniformly or generally uniformly distributed along the circumferential direction of the housing. For instance, the gap between the circuit board and the curved battery assembly and the gap between the first electrode and the second electrode divide the circumference of the housing into four generally equal segments.

The smart ring device provided in the present disclosure has a housing including an outer ring, an inner ring, and an accommodation space therebetween, at least one electronic component is arranged within the accommodation space, and the first and second electrodes electrically connected to the at least one electronic component are spaced apart circumferentially on the inner surface of the housing, enabling the monitoring of physiological parameters of the user using the first and second electrodes.

### BRIEF DESCRIPTION OF THE DRAWINGS

The accompanying drawings are incorporated into and constitute a part of the specification, illustrating embodiments or implementations consistent with the present disclosure, and are used in conjunction with the specification to explain the principles and implementations of the present disclosure.

FIG. 1 is a schematic structural diagram of a smart ring device according to some examples.

FIG. 2 is a disassembled schematic diagram of the smart ring device according to some examples.

FIG. 3 is another schematic structural diagram of the smart ring device according to some examples.

FIG. 4 is a schematic structural diagram of an outer ring of the smart ring device according to some examples.

FIG. 5 is another schematic structural diagram of the smart ring device according to some examples.

FIG. 6 is a schematic block diagram of the smart ring device according to some examples.

FIG. 7 is a schematic block diagram of charging the smart ring device according to some examples.

FIG. 8 is a schematic diagram of charging the smart ring device according to some examples.

FIG. 9 is a schematic diagram of manufacturing the smart ring device according to some examples.

FIG. 10 is another schematic diagram of manufacturing the smart ring device according to some examples.

FIG. 11 is another schematic diagram of manufacturing the smart ring device according to some examples.

### Description of reference numerals:

100: smart ring device, 10: housing, 11: outer ring, 111: inner surface of the outer ring, 112: first mounting region, 113: second mounting region, 1131: locating component, 1132: groove, 12: inner ring, 121: first aperture, 122: second aperture, 123: opening, 13: accommodation space, 20: electronic component, 21: galvanic skin response (GSR) sensing module, 22: charging interface, 23: temperature sensing module, 24: optical detection module, 241: convex lens, 242: first optical component, 243: second optical component, 2431: first light emitter, 2432: second light emitter, 244: third optical component, 30: first electrode, 31: support portion, 311: locating groove, 32: detection portion, 40: second electrode, 50: isolating component, 60: isolating ring, 70: circuit board, 71: rigid circuit board, 72: flexible circuit board, 721: mating component, 73: conductive wire, 80: battery assembly, 200: charging case, 201: charging terminal, 300: upper positioning jig, 400: lower positioning jig, 500: silicone jig.

### DETAILED DESCRIPTION

Example implementations will be described herein in detail with reference to the accompanying drawings. In the description referring to the drawings, a same numeral in different drawings denote a same or similar elements unless otherwise specified. The example implementations described in the following do not represent all implementations consistent with the present disclosure. Instead, they are merely examples of the methods and devices consistent with some aspects of the present disclosure as detailed in the appended claims.

The terminology used in the present disclosure is used for the purpose of describing particular implementations and is not intended to limit the present disclosure. Unless otherwise defined, technical or scientific terms used herein shall have the ordinary meaning as understood by one of ordinary skill in the art to which the present disclosure belongs. The terms "first", "second" and the like are used in the specification and claims of the present disclosure do not denote any order, quantity, or importance, but are used merely to distinguish different components. Similarly, terms such as "a" or "an" do not denote a quantity limitation, but rather denote the presence of at least one. The term "multiple" or "a plurality of' denotes two or more. The terms such as "comprises", "includes" and the like represent that an element or object preceding the terms encompass the elements or objects as well as their equivalents after the terms, without excluding other elements or objects. The terms "connected", "coupled" and the like are not limited to physical or mechanical connections, and may include electrical connections, which can be direct or indirect.. The singular forms "a", "an", and "the" used in the specification and appended claims of the present disclosure are also intended to include the plural forms, unless the context clearly indicates otherwise. It should be understood that the term "and/or" used herein refers to and includes any or all possible combinations of one or more of the associated listed items. The term "fitting" or "adapting" in the present disclosure includes, but is not limited to, the installation or removal relationship between elements or structures as well as the mounting or dismounting process.

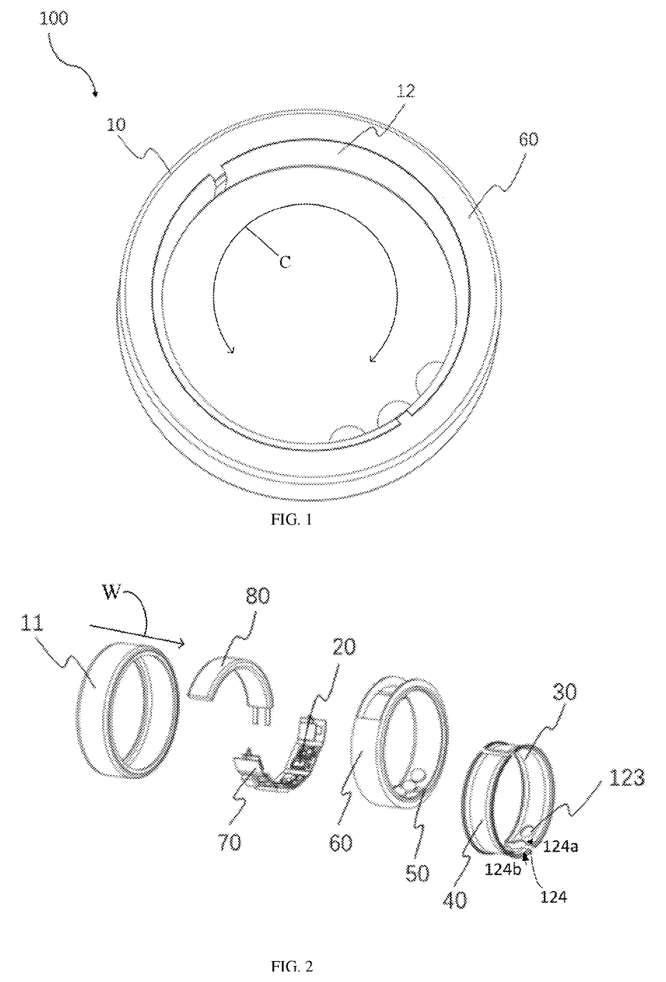

Referring to FIGS. 1 to 5, an example of a smart ring device 100 is shown. The smart ring device 100 includes a housing 10 and at least one electronic component 20. The housing 10 may be annular and includes an outer ring 11, an inner ring 12, and an accommodation space 13 located between the outer ring 11 and the inner ring 12. The at least one electronic component 20 is arranged within the accommodation space 13. The smart ring device 100 further includes a first electrode 30 and a second electrode 40. The first electrode 30 and the second electrode 40 are spaced apart from each other along the circumferential direction C (shown in FIG. 1) of the inner surface of the housing 10. The first electrode 30 and the second electrode 40 are electrically connected to the at least one electronic component 20.

In the smart ring device 100, the accommodation space is arranged between the outer ring 11 and the inner ring 12 of the housing 10. The at least one electronic component 20 is arranged within the accommodation space. The first electrode 30 and the second electrode 40, which are electrically connected to the at least one electronic component 20, are arranged along the circumferential direction of the inner surface of the housing 10. At least a portion of the first electrode 30 and at least a portion of the second electrode 40 are configured for contacting the finger skin of a user. Thus, when the smart ring device 100 is worn on a user's finger, at least a portion of the first electrode 30 and at least a portion of the second electrode 40 may contact the finger skin and obtain one or more physiological signals by the at least one electronic component 20 from the user, thereby realizing the health monitoring of the user based on the smart ring device 100.

The first electrode 30 and the second electrode 40 are configured to obtain one or more types of physiological signals, including at least one of an electrodermal activity (EDA) signal, a body impedance signal, a heart rate detection signal, a blood pressure signal, an electrocardiogram (ECG) signal, or a body temperature signal or the like.

In some examples, at least one of the first electrode 30 or the second electrode 40 is multiplexed (i.e., shared) to acquire multiple types of physiological signals, thereby reducing the number of electrodes in the smart ring device 100, and further streamlining the structure of the smart ring device and reducing device cost. In some examples, at least one of the first electrode 30 or the second electrode 40 is shared to acquire both an EDA signal and a body temperature signal. In some other examples, at least one of the first electrode 30 or the second electrode 40 are shared to acquire both an EDA signal and an ECG signal. In some other examples, while at least one of the first electrode 30 or the second electrode 40 are configured to acquire physiological signals, the first electrode 30 and the second electrode 40 are also served as charging terminals. For instance, while at least one of the first electrode 30 or the second electrode 40 is served as body temperature sensing electrodes, the first electrode 30 and the second electrode 40 are also served as charging terminals. The specific implementations of sharing the electrodes are not limited herein.

The housing 10 includes the outer ring 11 and the inner ring 12. The outer surface of the outer ring 11 may form at least a portion of the outer surface of the housing 10, and the inner surface of the inner ring 12 may form at least a portion of the inner surface of the housing 10.

In some examples, the outer ring 11 includes an electrically conductive material, such as a metallic material like titanium alloy, stainless steel, or aluminum alloy, etc. In an example, the outer ring 11 is made from titanium alloy, which provides the smart ring device 100 with a relatively high overall hardness and improved aesthetics.

In some examples, the inner ring 12 may be made from an electrically conductive material such as a metallic material, or made from an electrically insulating material such as a non-metallic material. Alternatively, a portion of the inner ring 12 includes an electrically conductive material such as a metallic material, and another portion of the inner ring 12 includes an electrically insulating material such as a non-metallic material. In some examples, the inner ring 12 is made from a non-conductive material, such as a moldable material like epoxy resin. In such cases, optionally, the inner ring 12 includes at least one aperture, and the first electrode 30 and the second electrode 40 protrude from the inner surface of the inner ring 12 through the at least one aperture. For instance, both the first electrode 30 and the second electrode 40 protrude from the inner surface of the inner ring 12 through a same aperture. For another instance, the first electrode 30 and the second electrode 40 protrude from the inner surface of the inner ring 12 through different apertures. Alternatively, at least a part of the first electrode 30 and the second electrode 40 are coated on the inner surface of the inner ring 12. For instance, at least one of the first electrode 30 or the second electrode 40 includes a metallic coating layer on the inner surface of the inner ring 12.

In some examples, the inner ring 12 includes a metallic material, which may be same or different from the metallic material of the outer ring 11. In such cases, at least a portion of the inner surface of the inner ring 12 may serve as the first electrode 30 and the second electrode 40, e.g., the first electrode 30 includes a first portion of the inner surface of the inner ring 12, and the second electrode 40 includes a second portion of the inner surface of the inner ring 12. Alternatively, at least one aperture is provided on the inner surface of the inner ring 12, and the first electrode 30 and the second electrode 40 protrude from the inner surface of the inner ring 12 through the at least one aperture. In such cases, an electrically insulating layer is optionally provided between at least one of the first electrode 30 or the second electrode 40 and the inner surface of the inner ring 12.

The accommodation space 13 is provided between the outer ring 11 and the inner ring 12. In some examples, at least a portion of a circumference wall defining the accommodation space 13 may include the outer ring 11 and the inner ring 12. In such cases, the outer ring 11 and the inner ring 12 enclose to form the accommodation space 13. Alternatively, one or more other components, such as other annular components, may be provided between the outer ring 11 and the inner ring 12, and accordingly, the circumference wall defining the accommodation space 13 may include one of the outer ring 11 and the inner ring 12.

In some examples, both the outer ring 11 and the inner ring 12 are made from metallic materials, or the inner ring 12 is made from non-metallic materials, and an isolation ring 60 including an electrically insulating material, such as one or more non-metallic materials (e.g., epoxy resin, polyurethane, polyethylene, etc.), may be provided between the outer ring 11 and the inner ring 12. The isolation ring 60 may be formed from potting or any other suitable processes. In such cases, the accommodation space 13 may be formed between the outer ring 11 and the isolation ring 60, that is, the outer ring 11 and the isolation ring 60 enclose to form the accommodation space 13. Alternatively, the accommodation space 13 may be formed between the inner ring 12 and the isolation ring 60, that is, the inner ring 12 and the isolation ring 60 enclose to form the accommodation space 13.

In an example, the accommodation space 13 may be defined by the outer ring 11 and the isolation ring 60, and the circumference wall of the accommodation space 13 may include at least a portion of the outer ring 11 and at least a portion of the isolation ring 60. In such cases, the isolation ring 60 may be optionally arranged on the outer surface of the inner ring 12. The isolation ring 60 may, for instance, include or be formed by a coating or a potted layer on the outer surface of the inner ring 12.

In another example, the accommodation space 13 may be defined by the inner ring 12 and the isolation ring 60, and the circumference wall of the accommodation space 13 may include at least a portion of the inner ring 12 and at least a portion of the isolation ring 60. In such cases, the isolation ring 60 is optionally arranged on the inner surface of the outer ring 11. The isolation ring 60 may, for instance, include or be formed by a coating or a potted layer on the inner surface of the outer ring 11.

The at least one electronic component 20 is arranged within the accommodation space 13. The at least one electronic component 20 may include at least one of a charging assembly or at least one physiological sensor. The physiological sensor may acquires physiological data based on physiological signals obtained by at least one electrode. The at least one physiological sensor may include at least one of a bio-electrical impedance analysis (BIA) sensor, a body temperature sensor, a electrocardiogram sensor, or an EDA sensor or the like. Additionally, at least one of a processor, a memory, a battery component, a communication component (e.g., wired and/or wireless communication module), a locating component or the like may be arranged or partially arranged within the accommodation space 13, which is not limited in the present disclosure.

In some examples, an optical detection module 24 is arranged within the accommodation space 13. The optical detection module 24 may be configured to detect a pulse or heart or blood related signal from the user to obtain one or more physiological parameters, such as heart rate, blood oxygen saturation, blood pressure, blood glucose or the like, or further to determine whether the one or more physiological parameters are abnormal and promptly alert the user when an abnormal condition is detected.

The optical detection module 24 may include a plurality of optical components, such as at least one light emitter and at least one light detector. A distance between the light emitter and the light detector may be in the range from 3 mm to 12 mm, which is not limited herein.

In some examples, the at least one light emitter may include one or more LEDs configured to emit light signals associated with a single wavelength, such as, green light, red light, or infrared light or the like. The at least one light emitter may include one or more LEDs configured to emit light signals associated with two wavelengths, such as red light and infrared light. The at least one light emitter may include one or more LEDs configured to emit light signals associated with three wavelengths, such as green light, red light, and infrared light. The light detector may include a photodiode (PD) configured to detect light signals associated with a certain wavelength range, such as 400 nm to 1000 nm. For instance, the optical detection module 24 may utilize green light for heart rate detection. For another instance, the optical detection module 24 may utilize red and infrared light for blood oxygen saturation detection.

In some examples, the optical detection module 24 may be a reflective or transmissive optical detection module. The light emitter and the light detector may be located on a same side relative to the finger skin. For instance, the optical emitter and the optical detector may be arranged adjacent to each other within the housing 10. Alternatively, the light emitter and the light detector may be located on different sides relative to the finger skin. For instance, the light emitter and the light detector may be located in opposite regions of the housing 10. The present disclosure does not set limitation on the implementations of the optical detection module 24.

In some examples, the at least light emitter may be configured to emit light signals. The emitted light signals may be received by the light detector after being incident on the finger skin and reflected and/or refracted by the finger skin. After the light signals received by the light detector are analyzed and processed, a physiological measurement may be obtained.

In some examples, to enable the light signals emitted from the light emitter to be incident on the finger skin and to allow the light signals returning from the finger skin to be received by the light detector, the inner ring 12 includes one or more light-transmitting regions in correspondence with the at least one light emitter and the at least one light detector, as well as a light-blocking region between the light-transmitting regions.

In an example, the inner ring 12 may include a light-transmitting material, such as glass, sapphire, epoxy resin, transparent plastic or the like, and a light-blocking layer may be provided at locations not corresponding to the at least one light emitter and the at least one light detector, so as to block the direct light passage between the light emitter and the light detector and improve aesthetics of the smart ring device. For instance, an ink layer may be provided on the surface of the inner ring. The ink layer includes ink covers areas not corresponding to the optical components (i.e., at least one light emitter and at least light detector), that is, ink covers regions between the optical components, and apertures corresponding to the optical components. Alternatively, an additional light-shielding structure, such as a light-shielding bracket, may be provided between the inner ring 12 and the optical detection module 24, and the bracket includes corresponding light-transmitting and light-shielding regions.

In another example, the inner ring 12 may include an opaque material, and at least one aperture is provided at positions corresponding to the at least one light emitter and the at least one light detector, with at least one light-transmitting lens arranged within the at least one aperture to form one or more light-transmitting regions. The bracket may be mounted on the outer surface of the inner ring 12, but the present disclosure is not limited thereto.

In some examples, the inner ring includes a light-transmitting material that allows light emitted and received by the optical detection module to pass through.

In some examples, the optical detection module 24 may further include at least one convex lens 241 corresponding to the plurality of optical components. The convex lens 241 may be elliptical or any other shapes to converge light signals, which in turn is conductive to reduce the power consumption of the optical detection module 24. Additionally, the convex lens 241 may serve as a part of the inner surface of the housing, enhancing the tightness of contact with finger skin to improve measurement accuracy. The convex lens 241 may be also configured as a positional guide during wearing or charging of the smart ring device 100. For instance, when the smart ring device 100 is placed in the charging case 200 for charging, the convex lens 241 may mate with guide grooves on the charging case 200, achieving the effect of locating and anti- misalignment, and improving charging safety. In the example shown in FIG. 2, the convex lens has a shape of an elliptical racetrack, and is formed from a light-transmitting material, such as transparent silicone, or epoxy resin, or the like.

In an example, the inner ring 12 includes at least one opening 123, and a convex lens 241 protruding from the inner surface of the inner ring 12 may be arranged within the opening 123. In another example, at least one convex lens 241 may be arranged within the accommodation space 13, and the inner ring 12 may include an opening 123 at a corresponding position to allow the convex lens 241 to extend from the opening 123. In yet another example, the inner ring 12 includes a light-transmissive material and has at least one convex protrusion at a position corresponding to the optical components, and the at least one convex protrusion may be configured as the convex lens 241.

In some examples, the inner surface of the housing 10 includes at least one convex lens 241 at positions corresponding to the optical components. Specifically, at least one opening 123 may be defined on the inner ring 12, and the at least one convex lens 241 is embedded in the at least one opening 123 and protrudes from the inner surface of the inner ring 12. For instance, at least one convex lens 241 is arranged in the accommodation space 13, and an opening 123 is provided at an corresponding position of the inner ring 12 to allow the convex lens 241 to protrude from the opening 123. In some other examples, the inner ring 12 may include light- transmitting material and have at least one convex protrusion at positions corresponding to the optical components, and the at least one convex protrusion may serve as the convex lens 241. In other words, the inner surface of the inner ring 12 may include at least one convex protrusion for converging light signals.

In some examples, to prevent light signals emitted by the light emitter from directly entering the light detector and causing crosstalk, the optical detection module 24 may further include an optical barrier arranged between the at least one light emitter and the at least one light detector. For instance, a first end of the optical barrier is connected to the circuit board on which the optical components are provided, and a second end of the optical barrier is connected to the surface (i.e., the outer surface or the inner surface) of the inner ring 12, but no limitation is set on the specific implementations of the optical barrier.

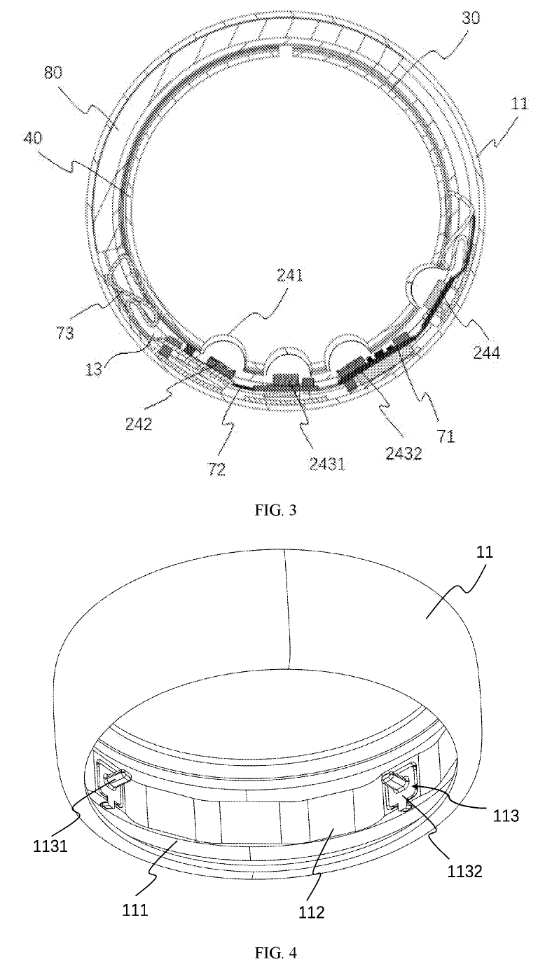

In some examples, referring to FIGS. 2 and 3, both the first electrode 30 and the second electrode 40 are in the shape of semi-annular or substantially semi-annular. The first electrode 30 and the second electrode 40 may enclose to form an annular or generally annular configuration along the circumferential direction of the inner surface of the inner ring 12. In such cases, the first electrode 30 and the second electrode 40 may have a relatively large surface area, and correspondingly, have a relatively large contact area with the finger skin, which is beneficial for improving the efficiency and accuracy of physiological detection.

In some examples, the first electrode and the second electrode collectively extend over more than two-thirds of the circumference of the inner ring 12. In some examples, at least one of the first electrode 30 or the second electrode 40 occupies a dimensional ratio exceeding two-thirds along the width direction (W, as shown in FIG. 2) of the inner ring 12. In some other examples, the first electrode 30 and the second electrode 40 occupy more than two-thirds of the surface area on the inner surface of the inner ring 12. In such cases, the first electrode 30 and the second electrode 40 have a relatively large contact area with the finger skin of the user.

In some examples, referring to FIG. 2, the inner ring 12 includes the first electrode 30, the second electrode 40, and an isolation component 50 arranged between the first electrode 30 and the second electrode 40 along the circumferential direction of the inner ring 12. The isolation component 50 includes an electrically insulating material such as a non-metallic material. This configuration ensures that both the first electrode 30 and the second electrode 40 have a sufficiently large contact area with the finger skin, which is beneficial for the smart ring device 100 to accurately and efficiently obtain signals through the first electrode 30 and the second electrode 40. Meanwhile, the isolation component 50 provides electrical isolation between the first electrode 30 and the second electrode 40, and also provides an antenna clearance area. In the example shown in FIG. 2, two isolation components 50 are provided in the gap between the first electrode 30 and the second electrode 40, located at the connection portions of the first electrode 30 and the second electrode 40, respectively. However, in some other examples, the number of isolation components 50 may be more or less, which is not limited in the present disclosure.

In some examples, the first electrode 30 and the second electrode 40 may be identical electrodes, which ensures signal acquisition performance and facilitates manufacturing and processing. In some other examples, the first electrode 30 and the second electrode 40 have different configurations, which is not limited in the present disclosure.

Furthermore, in some examples, at least one of the first electrode 30 or the second electrode 40 includes at least one opening 123. The at least one opening 123 may be arranged corresponding to the plurality of optical components included in the optical detection module 24 disposed within the accommodation space 13, allowing the plurality of convex lenses 241 corresponding to the plurality of optical components to protrude through the at least one opening 123.

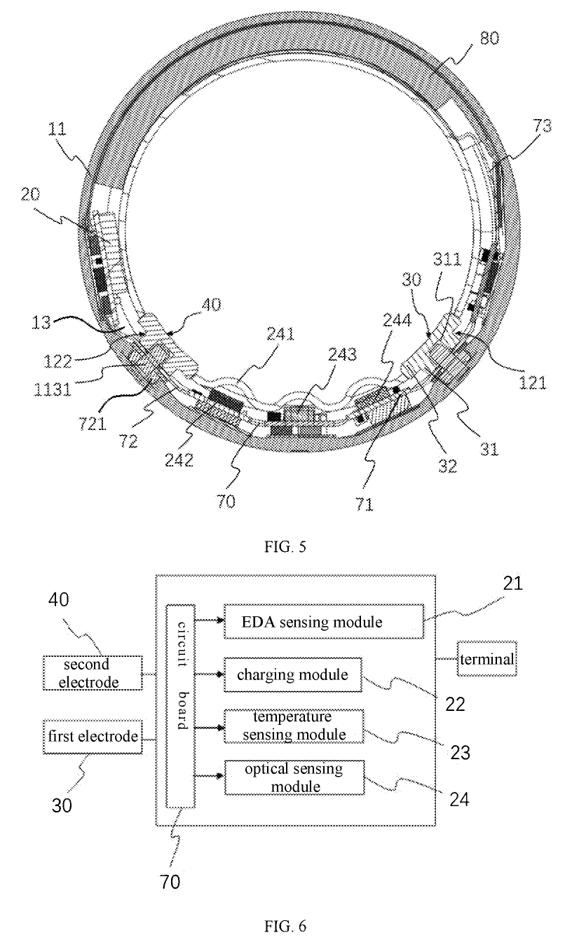

In the examples shown in FIGS. 2 and 5, three optical components are arranged within the accommodation space 13 along the circumferential direction of the housing 10, namely a first optical component 242, a second optical component 243, and a third optical component 244. The second optical component 243 is located in a region where the isolation component 50 connects the first electrode 30 and the second electrode 40. The first optical component 242 and the third optical component 244 are arranged on opposite sides of the second optical component 243.

In an example, the first optical component 242 and the third optical component 244 are symmetrically arranged with respect to the second optical component 243. Each of the first optical component 242, the second optical component 243, and the third optical component 244 may be a light emitter or a light detector. In some examples, the second optical component 243 is a light emitter, and the first optical component 242 and the third optical component 244 both are light detectors. In some other examples, the second optical component 243 is a light detector, and the first optical component 242 and the third optical component 244 are both light emitters. In some other examples, the number of optical components may be more or less, and the optical components may be arranged in other configurations, which is not limited in the present disclosure. An example is described herein, where the second optical component 243 is a light emitter and both the first optical component 242 and the third optical component 244 are light detectors. The second optical component 243 and the first optical component 242 may form a first optical detection channel. The second optical component 243 and the third optical component 244 may form a second optical detection channel. In some examples, in the case where the second optical component 243 is an LED for emitting light signals associated with two wavelengths or an LED for emitting light signals associated with three wavelengths, the second optical component 243 may include more than two light-emitting elements, each of which may form different optical detection channels with the first optical component 242 and/or the third optical component 244, which is not limited in the present disclosure.

In the example shown in FIG. 3, the optical detection module 24 includes four optical components: a first light emitter 2431, a second light emitter 2432, a first light detector (the first optical component 242), and a second light detector (the third optical component 244). The first light emitter 2431 and the second light emitter 2432 are arranged near the region where the isolation component 50 connects the first electrode 30 and the second electrode 40. The first light detector (the first optical component 242) and the second light detector (the third optical component 244) are arranged on opposite sides of the first light emitter 2431 and the second light emitter 2432. The first light emitter 2431 and the second light emitter 2432 are configured to emit visible light or infrared light, and together with the first light detector (the first optical component 242) and the second light detector (the third optical component 244), forming at least one optical detection channel for obtaining physiological signals. In an example, a distance between the first light emitter 2431 and the first light detector (the first optical component 242) is 4 mm, forming a first optical detection channel. A distance between the first light emitter 2431 and the second light detector (the third optical component 244) is 12 mm, forming a second optical detection channel. A distance between the second light emitter 2432 and the first light detector (the first optical component 242) is 8 mm, forming a third optical detection channel. A distance between the second light emitter 2432 and the second light detector (the third optical component 244) is 8 mm, forming a fourth optical detection channel. The plurality of optical components form four optical detection channels, enabling a relatively high quality of measurement signals and accurate physiological parameters. Similarly, in some examples, in the case where the first light emitter 2431 or the second light emitter 2432 is the LED for emitting light signals associated with two wavelengths or an LED for emitting light signals associated with three wavelengths, more optical detection channels may be formed, or each optical detection channel may include more optical components, which is not limited in the present disclosure.

Furthermore, in some examples, a convex lens 241 is correspondingly provided for each optical component. In the example shown in FIG. 2, three openings are provided on the smart ring device 100. The first electrode 30 and the second electrode 40 may each include an opening 123 (only shown for the first electrode 30 in this example), which can be a full or complete opening. The opening 123 can be located away from the ends, For instance, in an intermediate region of either of the first electrode 30 or the second electrode 40, respectively. Another opening, e.g., an opening 124, may be formed by a half opening 124a at an end of the first electrode 30 and a half opening 124b at an end of the second electrode 40. The half opening 124a of the first electrode 30 and the half opening 124b of the second electrode 40 may together form a full or complete opening after assembly of the smart ring device 100. In such cases, for instance, the two convex lenses 241 corresponding to the first optical component 242 and the third optical component 244 respectively protrude through the two complete openings 123 provided on the first electrode 30 and the second electrode 40, respectively, and the convex lens 241 corresponding to the second optical component 243 protrudes through the opening 124 formed by assembling (or combination) of the first electrode 30 and the second electrode 40.

Furthermore, in the example shown in FIG. 2, the isolation ring 60 is provided between the outer ring 11 and the inner ring 12, and the accommodation space 13 is arranged between the isolation ring 60 and the outer ring 11. The isolation ring 60 may include a non- metallic material such as silicone or resin. At least one of the convex lens 241 or the isolation component 50 may be arranged on the isolation ring 60. The outer ring 11 and the inner ring 12 are respectively secured to opposite sides (i.e., the inner side and the outer side) of the isolation ring 60. This arrangement allows the isolation ring 60 to provide electrical insulation and improve the overall aesthetics of the smart ring device 100. The accommodation space 13 provides a receiving space for the one or more electronic components 20, enabling rational utilization of the space within the smart ring device 100 and realization of various functions.

In some other examples, as shown in FIG. 5, a first aperture 121 and a second aperture 122 are symmetrically provided along the circumferential direction of the inner surface of the inner ring 12, such as with respect to the second optical component 243. The first electrode 30 protrudes from the inner surface of the inner ring 12 through the first aperture 121, and the second electrode 40 protrudes from the inner surface of the inner ring 12 through the second aperture 122. In such cases, the inner surface (or inner wall) of the inner ring 12 may include a non-metallic material, or, an electrically insulating layer may be provided between the inner surface (or inner wall) of the inner ring 12 and the first and second electrodes 40. The first electrode 30 and the second electrode 40 are arranged on opposite sides of the optical detection module 24, e.g., the first electrode 30 and the second electrode 40 are arranged circumferentially surrounding the optical components 242, 243, and 244. In such cases, a plurality of convex lenses 214 may be arranged on the inner surface (or inner wall) of the inner ring 12. For instance, the inner surface of the inner ring 12 includes a plurality of convex protrusions serving as the convex lenses 214, but the present disclosure is not limited thereto.

In some examples, the smart ring device 100 further includes a circuit board 70 arranged within the accommodation space 13. One or more electronic components 20 are disposed on the circuit board 70. Alternatively, one or more other components, such as at least one of the optical detection module 24, a temperature sensing module 23, a charging interface 22, an electrodermal activity sensing module 21, or a communication module, may be disposed on the circuit board 70. A first end of each of the first electrode 30 and the second electrode 40 may be arranged on the circuit board 70, and a second end of each of the first electrode 30 and the second electrode 40 protrudes from the inner surface of the inner ring 12. In such cases, both the first electrode 30 and the second electrode 40 may have a certain thickness. For instance, as shown in FIG. 5, the first electrode 30 and the second electrode 40 may include a support portion 31 and a detection portion 32. The support portion 31 is arranged on the circuit board 70, and the detection portion 32 protrudes from the inner surface of the inner ring 12. The support portion 31 may be secured to the circuit board 70, For instance, via adhesive, locating pins, screws, etc. Alternatively, the support portion 31 may be secured to the outer ring 11. For instance, as shown in FIG. 5, the support portion 31 includes a locating groove 311, and a locating component 1131 on the outer ring 11 is disposed in the locating groove 311, enabling positioning of the first electrode 30 and the second electrode 40. To make the positions of the first electrode 30 and the second electrode 40 more stable, in some examples, adhesive or other fastening components may be provided at the contact region between the locating component 1131 and the detection portion 32, which is not limited herein.

Referring to FIG. 3 and FIG. 5, the smart ring device 100 further includes the circuit board 70 arranged within the accommodation space 13. The circuit board 70 includes at least two rigid circuit boards 71 and a flexible circuit board 72 connecting the at least two rigid circuit boards 71, and at least one electronic component 20 is arranged on the at least two rigid circuit boards 71.

As shown in FIGS. 3 and 5, to adapt to the annular structure of the smart ring device 100 and mount as many electronic components 20 as possible on the circuit board 70, the circuit board 70 may be configured in a curved shape. The circuit board 70 may include one or more rigid circuit boards 71 and one or more flexible circuit boards 72. For instance, the circuit board 70 includes at least two rigid circuit boards 71 and at least one flexible circuit board 72 connecting the at least two rigid circuit boards 71. The rigid circuit board 71 may be, for instance, a printed circuit board (PCB). The one or more electronic components 20 may be arranged on the at least two rigid circuit board 71 to ensure their stability. The flexible circuit board 72 may be, for instance, a flexible printed circuit (FPC). The flexible circuit board 72 is configured to connect adjacent rigid circuit boards 71 to form a curvature adapted to the annular shape (i.e., the shape of the accommodation space 13) of the smart ring device 100.

In some examples, the curvature of the flexible circuit board 72 may adapt to the receiving space (i.e., accommodation space 13) of the smart ring device 100, enabling a same circuit board 70 to be adapted to smart ring devices with different sizes and configurations, thereby achieving the versatility of the circuit board 70. The circuit board 70 may be, for instance, a rigid-flex circuit board. By adjusting the curvature of the flexible circuit board 72 and the angle between adjacent rigid circuit boards 71 according to the shape and size of the smart ring device 100, the curvature and overall length of the rigid-flex circuit board along the circumference of the smart ring device 100 may be changed to match the size and shape of the smart ring device 100, without requiring additional mechanisms and parts.

In some examples, a reinforcing plate, such as a steel stiffener, may be provided at the flexible circuit board 72 to enhance the structural strength of the flexible circuit board 72, which is not limited in the present disclosure.

In some examples, all of the at least one electronic component 20 in the smart ring device 100 may be arranged on the rigid circuit board 71, while the flexible circuit board 72 is free of the electronic component 20 and only provided with necessary connection terminals or connectors.

In some other examples, a first subset of the at least one electronic component 20 in the smart ring device 100 may be arranged on one or more rigid circuit boards 71, and a second subset of the at least one electronic component 20 may be arranged on one or more flexible circuit boards 72.

In yet some other examples, one or more additional electronic components of the smart ring device 100 may be arranged on the rigid circuit board 71 or the flexible circuit board 72. For instance, the at least one electronic component 20 is arranged on the rigid circuit board 71, and one or more additional electronic components are arranged on the flexible circuit board 72. For another instance, all of the at least one electronic component 20 and the additional electronic components are disposed on the rigid circuit board 71, but the present disclosure is not limited thereto.

The first electrode 30 and the second electrode 40 may be electrically connected to the circuit board 70, for instance, the rigid circuit board 71 and/or the flexible circuit board 72. In some examples, the circuit board 70 may be electrically connected to the first electrode 30 and the second electrode 40 through a wire 73 or other types of electrical connection mechanisms. The first electrode 30 and the second electrode 40 may be electrically connected to the rigid circuit board 71 or the flexible circuit board 72.

In some examples, a bonding pad may be reserved on the rigid circuit board 71 or the flexible circuit board 72, and the first electrode 30 and the second electrode 40 are soldered to the bonding pad by surface mount technology (SMT). For instance, one end of the wire 73 may be soldered to the first electrode 30 and/or the second electrode 40, and the other end of the wire 73 is connected to the rigid circuit board 71 or the flexible circuit board 72.

In some examples, a metal piece may be arranged within the accommodation space 13 of the housing 10, and the connection between the outer ring 11, the inner ring 12, or the circuit board 70 and the first electrode 30 and/or the second electrode 40 are achieved by soldering to the metal piece. Alternatively, conductive adhesive may be used to bond the circuit board 70 to the first electrode 30 and/or the second electrode 40, establishing the electrical connection therebetween.

The optical detection module 24 may be arranged on the rigid circuit board 71 or the flexible circuit board 72. In some examples, the area of the circuit board 70 for mounting the optical components may include an opaque material, to prevent light to pass through, may be provided at the area of the circuit board 70 for disposing optical devices. For instance, the region of the circuit board 70 for mounting optical components may include light-impermeable material or a light-shielding layer to avoid light crosstalk between different optical components. For instance, the region on the circuit board 70 between the light emitter and the light detector may include an opaque component.

In some other examples, the first optical component 242 and the third optical component 244 may be encapsulated on the rigid circuit board 71 with a light-transmitting material, which allows light to pass through, such as silicone or resin, and an opaque optical barrier may be arranged around the second optical component 243. Alternatively, an opaque optical barrier may be arranged around the first optical component 242 and the third optical component 244, respectively, which is not limited in the present disclosure.

The circuit board 70 may be secured within the accommodation space 13 of the smart ring device 100 by a variety of locating structures, such as adhesive, locating components, welding or the like. In some examples, as shown in FIG. 4, at least one of a first mounting region 112 or a second mounting region 113 is provided on the inner surface 111 of the outer ring. At least a portion of the rigid circuit board 71 is secured to the first mounting region 112 by a first locating structure, and at least a portion of the flexible circuit board 72 is secured to the second mounting region 113 by a second locating structure different from the first locating structure. In some examples, the first locating structure may include adhesive or welding, and the second locating structure may include a locating component; or vice versa. For instance, the second mounting region 113 includes a locating component 1131, and the flexible circuit board 72 includes a mating component 721 engaging the locating component 1131. The locating component 1131 may include a locating post (i.e., locating pin), and the mating component 721 may include a locating aperture. Alternatively, the locating component may include a screw, and the mating component 721 may include a nut, etc. In this way, the rigid circuit board 71 and the flexible circuit board 72 may be secured in the accommodation space 13 respectively by different locating structures, improving the locating effectiveness of the circuit board 70.

Additionally, by adapting and connecting the mating component 721 on the flexible circuit board 72 with the locating component 1131, the flexible circuit board 72 may be secured to the outer ring 11, facilitating fixing the circuit board 70 to the outer ring 11, preventing the circuit board 70 from moving relative to the outer ring 11, ensuring the functional stability of the smart ring device 100, and improving assembly convenience.

In some examples, to further improve the locating effect, as shown in FIGS. 4 and 5, a groove 1132 is provided on the second mounting region 113. One end of the locating component 1131 is fixedly disposed within the groove 1132, and the other end of the locating component 1131 is connected to the first electrode 30 or the second electrode 40 through the mating component 721. Thus, the locating component 1131 not only serves to position the circuit board 70, but also further assist in positioning the electrode. The locating component 1131 may be fixed within the groove 1132 means such as adhesive dispensing or snap-fit engagement or the like, and connected to the first electrode 30 or the second electrode 40 by means such as adhesive dispensing, soldering, or snap-fit engagement or the like. As shown in FIG. 5, a locating groove 311 may be provided on the first electrode 30 and/or the second electrode 40. The end portion of the locating component 1131 may be arranged within the locating groove 311 of the electrode, and fixed thereto by means such as adhesive dispensing or snap-fit engagement or the like.

In some examples, the locating component includes a locating pin, and the mating component includes a locating aperture.

In some examples, the locating pin may be connected to the first electrode or the second electrode after passing through the locating aperture, such as by abutting, gluing, welding or the like.

Referring to the example of FIG. 5, the outer ring 11 of the smart ring device 100 includes a locating component 1131, and the circuit board 70 includes a locating aperture adapted to the locating component 1131. One end of the locating component 1131 is fixedly secured to the outer ring 11, and the other end of the locating component 1131 passes through the locating aperture on the circuit board 70 and connects to the locating groove 311 on the first electrode 30 or the second electrode 40. This configuration allows simultaneous fixation of both the circuit board 70 and the electrode through the adaptive locating structure including the locating component and the mating component.

In some examples, referring to FIGS. 3 and 5, the smart ring device 100 further includes the curved battery assembly 80. The battery assembly 80 may be semi-annular or generally semi-annular in shape. The battery assembly 80 and the circuit board 70 are arranged in different regions along the circumferential direction within the accommodation space 13. For instance, the curved battery assembly 80 and the circuit board 70 may enclose a shape adapted to the housing 10, such as in an annular or substantially annular shape.

In some examples, there may be at least one first gap between the curved battery assembly 80 and the circuit board 70. For instance, the curved battery assembly 80 and the circuit board 70 are completely non-overlapping (i.e., there is no overlap between the battery assembly 80 and the circuit board 70). In the examples shown in FIGS. 2 and 3, the curved battery assembly 80 and the circuit board 70 may be arranged in a displaced manner relative to the first electrode 30 and the second electrode 40, such that the at least one first gap between the curved battery assembly 80 and the circuit board 70 does not overlap with at least one second gap between the first electrode 30 and the second electrode 40 along the circumferential direction of the housing 10. This configuration allows more efficient utilization of the accommodation space 13, improves the compactness of the internal structure of the smart ring device 100, and reduces the thickness of the smart ring device 100. In some examples, the at least one first gap between the curved battery assembly 80 and the circuit board 70 and the at least one second gap between the first electrode 30 and the second electrode 40 are uniformly distributed along the circumferential direction of the housing 10. For instance, as shown in FIG. 2, the curved battery assembly 80 and the circuit board 70 are spaced apart vertically, i.e., the battery assembly 80 and the circuit board 70 are arranged in the upper and lower portions of the housing 10, respectively. The first electrode 30 and the second electrode 40 are spaced apart horizontally, i.e., the first electrode 30 and the second electrode 30 are arranged in the left and right portions of the housing 10, respectively. Therefore, a line connecting the two first gaps and a line connecting the two second gaps are perpendicular or approximately perpendicular to each other, but the present disclosure is not limited thereto.

In some examples, different components (or assemblies) of a same module may be arranged on a same circuit board, or different components of a same module may be arranged on different circuit boards. For instance, the first electrode 30 and the second electrode 40 may be arranged on different circuit boards. For another instance, the light emitter and the light detector of the optical detection module 24 may be arranged on different circuit boards, or different light emitters and/or different light detectors of the optical detection module 24 may be arranged on different circuit boards. In an example, the one or more light emitters of the optical detection module 24 may be arranged on a same circuit board, while different light detectors may be arranged on different circuit boards, which are different from the circuit board to which the light emitters are mounted. In another example, each individual optical component of the optical detection module 24 may be arranged on a separate circuit board, e.g., each light emitter of the LED is arranged on a separate circuit board, and each light detector (e.g., photo detector) is arranged on a separate circuit board, which is not limited in the present disclosure.

In some examples, the at least one electronic component 20 may be a single electronic component, or may include a plurality of electronic components that operate together, each implementing a part of the functionality described herein.

In some examples, the at least one electronic component 20 may include an electrode- based charging interface or one or more physiological sensors. As shown in the example of FIG. 6, the at least one electronic component 20 may include at least one of an electrodermal activity (EDA) sensing module 21 or a body composition sensing module, which is electrically connected to the first electrode 30 and the second electrode 40. Additionally, the accommodation space 13 may further include at least one of a charging interface 22 or a temperature sensing module 23. Optionally, at least one of the charging interface 22 or the temperature sensing module 23 shares the first electrode 30 and the second electrode 40 with at least one of the EDA sensing module 21 or the body composition sensing module. In such cases, when the smart ring device 100 is worn on a finger, the first electrode 30 and the second electrode 40 contact the finger skin to obtain signals and transmit the obtained signals to one or more sensing modules arranged on the circuit board 70. The one or more sensing modules process the signals to obtain physiological detection data or processed signals for further processing.

In some examples, the smart ring device 100 may further include a processing module such as a controller and a switching circuit. The switching circuit is configured to switch a connection state between the first electrode 30 and/or the second electrode 40 and various modules, enabling the first electrode 30 and the second electrode 40 to measure different types of physiological data, or to switch between charging and physiological measurement. The controller is configured to send control signals to the switching circuit.

In some other examples, instead of including processing means such as a controller, the smart ring device 100 may include a wireless communication module, such as a short- distance communication module, a Bluetooth communication module, a WIFI communication module, a cellular communication module, etc. The wireless communication module may receive control signals sent by a terminal device or a server, and the switching circuit may switch the connection status between the first electrode 30 and/or the second electrode 40 and various sensing module under the control of the control signals. Additionally, after obtaining the physiological signals and/or processed data, the smart ring device 100 may send the physiological signals and/or processed data to the terminal device or the server through the wireless communication module, so that the terminal device or the server performs further processing on the physiological signal and/or processed data to obtain the physiological detection result, and/or output the received data and/or physiological detection result to the user.

The terminal device may include a mobile device such as a mobile phone, a wearable device such as a smart wristwatch, a tablet computer, a vehicle-mounted terminal or the like. The terminal device may output the physiological detection result via audio, haptic, a display, etc., so that the user can view his or her health data on the terminal device, such as emotional state information, physiological stress information, electrocardiogram information, body composition information, body temperature, heart rate, blood oxygen saturation, etc. Alternatively, the terminal device or the server may obtain an insight and/or a health improvement suggestion based on the physiological signal and/or processed data and output the insight and/or health improvement suggestion to the user. Optionally, the user may interact with the terminal device to instruct the smart ring device 100 to perform specific operations or functions.

In some examples, the EDA sensing module 21 may be configured to detect, via the first electrode 30 and the second electrode 40, electrodermal activity signals indicating physiological and emotional changes of the user. The EDA sensing module 21 may transmit the obtained electrodermal activity signals to the terminal device or the server through the wireless communication module. Alternatively, one or more processing may be performed on the electrodermal activity signals before being transmitted to the terminal device or the server. For instance, the processing module of the smart ring device 100 may process the electrodermal activity signals, such as pre-processing, feature extraction, and/or the like, to obtain emotion- related feature data, and send the obtained feature data to the terminal device or the server for further processing, so as to obtain an emotion monitoring result. Alternatively, the processing module of the smart ring device 100 may process the electrodermal activity signals to obtain an emotion monitoring result indicating whether the user has an emotional response, and optionally send the emotion monitoring result to the terminal device or the server for output to the user, or for subsequent analysis and statistics.

In some other examples, the smart ring device 100 may further include an output module such as at least one of an audio output module, a haptic output module, or a display or the like. Correspondingly, after the processing module obtains the emotion monitoring result, the emotion monitoring result may be output to the user through the output module.

The emotion monitoring result may indicate whether the user has an emotional response, such as excitement, calmness, anger, or sadness, etc. In some examples, the detected emotional responses may be accumulated to determine an emotional tendency or emotional summary of the user over a longer period, and the emotional tendency or emotional summary may be optionally output to the user. Alternatively, a guidance or suggestion for improving the emotion of the user is further output. The guidance or suggestion may be or include, for instance, engaging in one or more physical activities and/or mental activities, which is not limited in the present disclosure.

In some examples, the body composition sensing module may be electrically connected to the first electrode 30 and the second electrode 40, and obtain the body composition signals from the user through the first electrode 30 and the second electrode 40. Based on the body composition signals, a body composition measurement result of the user, such as a body fat measurement result or the like, may be obtained. The body composition sensing module may be based on a two-electrode mode, a four-electrode mode, an eight-electrode mode, etc. In an example, the smart ring device 100 may further include one or more additional electrodes (e.g., a third electrode) for body composition measurement, e.g., one or more additional electrodes may be provided on the outer surface of the outer ring 11. When the user wears the smart ring device 100 on a finger, the first electrode 30 and the second electrode 40 contact the skin of the finger wearing the smart ring device 100, and the one or more additional electrodes may contact an adjacent finger of the user, or contact the other hand of the user, thereby realizing body composition measurement based on the four-electrode or eight-electrode mode. In another example, the smart ring device 100 may perform the body composition measurements in conjunction with other electronic devices, such as a wrist-worn device like a wristwatch, or a terminal device. For instance, the wristwatch includes at least one third electrode, and when the user wears both the smart ring device 100 and the wristwatch, or a finger of the user simultaneously contacts the first electrode 30 and the second electrode 40 on the smart ring device, and meanwhile the user contacts at least one third electrode on the wristwatch, the body composition measurement of the user may be achieved. In such cases, the smart ring device 100 may send the detected body composition signals, or signals obtained after performing one or more processing on the detected body composition signals, to another electronic device, such as the other device collaborating with the smart ring device 100 in the body composition measurement, such that the another electronic device obtains the body composition measurement result of the user based on the body composition signals received from the smart ring device and the body composition signals detected itself. Alternatively, the smart ring device 100 and the another electronic device may both transmit the detected body composition signals or signals obtained after one or more processing performed on the detected body composition signals to the terminal device or the server, so that the terminal device or the server obtains the body composition measurement result of the user based on signals received from the devices.

In some examples, the temperature sensing module 23 may be electrically connected to at least one of the first electrode 30 or the second electrode 40. The first electrode 30 and/or the second electrode 40 may conduct skin temperature to the temperature sensing module 23. The temperature sensing module 23 may acquire the temperature signals and transmit the temperature signals or signals obtained after performing one or more processing on the temperature signals to the terminal device or the server, so that the terminal device or the server obtains a body temperature monitoring result of the user. The body temperature monitoring result may include a monitoring result of the skin temperature or the core body temperature of the user. Thus, when the terminal device or the server detects an abnormal body temperature from the user, the terminal device may alert the user, further enhancing health monitoring effect for the user, which is beneficial for timely and effective health monitoring and early detection of abnormal health conditions.

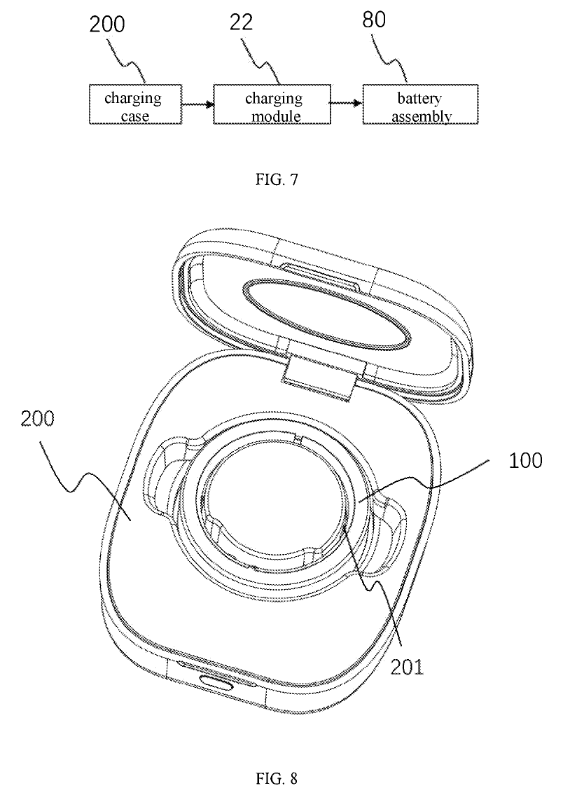

In some examples, the charging interface 22 may be electrically connected to the first electrode 30 and the second electrode 40, receive electrical power through the first electrode 30 and the second electrode 40, and provide the electrical power to the battery assembly 80. As shown in the examples of FIGS. 7 and 8, a charging case 200 may include a base and a charging portion. When the smart ring device 100 is placed in the charging case 200, the first electrode 30 and the second electrode 40 are correspondingly connected to a plurality of charging terminals 201 of the charging portion, enabling the charging case 200 to charge the smart ring device 100. The charging interface 22 may be based on wired charging or wireless charging, which is not limited in the present disclosure.

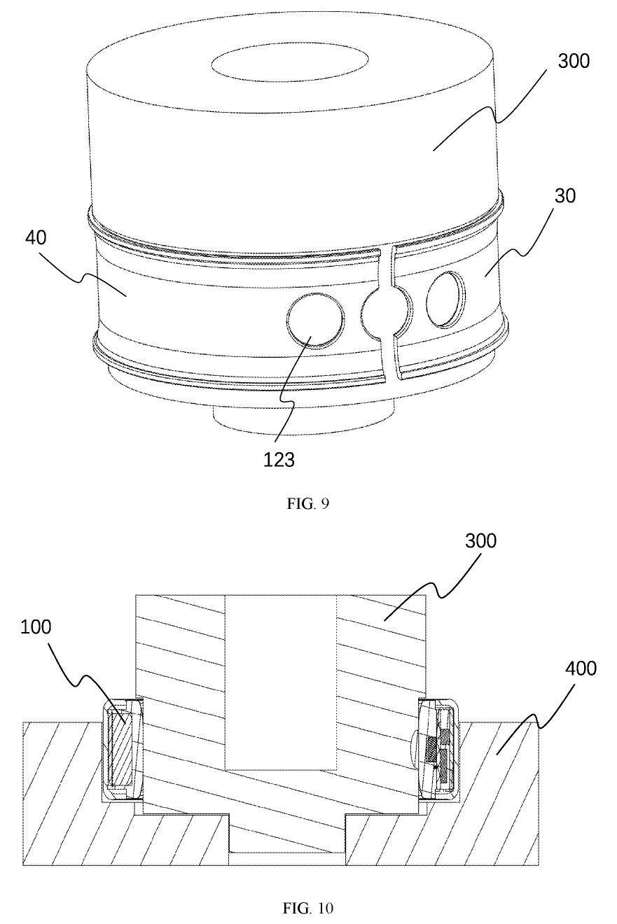

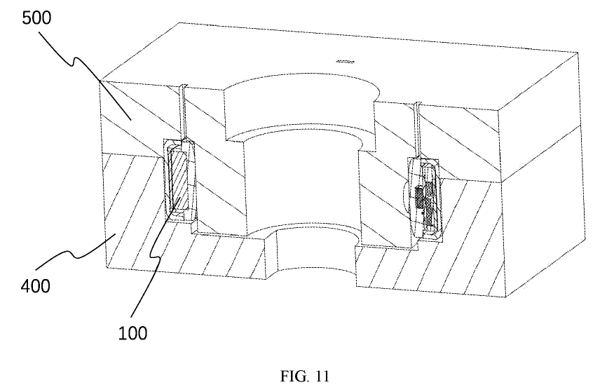

In some examples, the smart ring device 100 may be manufactured by potting and/or molding procedure. For instance, the assembly and manufacturing of the smart ring device 100 may be achieved through a jig. As shown in the examples of FIGS. 9 to 11, a jig may include an upper positioning jig 300, a lower positioning jig 400, and a silicone jig 500. The manufacturing of the smart ring device 100 may be achieved through a two-stage potting procedure.

First, the first electrode 30 and the second electrode 40 may be provided, for example, by vacuum suction with the upper positioning jig 300. The upper positioning jig 300 with the first electrode 30 and the second electrode 40 is placed into the lower positioning jig 400, so as to achieve positioning of the bottom portion of the first electrode 30 and the bottom portion of the second electrode 40. After the bottom portion of the first electrode 30 and the bottom portion of the second electrode 40 are positioned by using the upper positioning jig 300 and the lower positioning jig 400, adhesive dispensing is performed to bond the first electrode 30 and the second electrode 40 together. The bonding may be optionally achieved by potting of adhesive material.

In some other examples, the first electrode 30 and the second electrode 40 may be assembled into an integral unit by an NMT injection molding procedure, positioned using one or more jigs, and then bonded via potting of adhesive or the like.

Next, the circuit board 70, the curved battery assembly 80 and one or more electronic components 20 may be assembled to the outer ring 11. The wire 73 is soldered to the first electrode 30 and the second electrode 40.

Finally, the silicone jig 500 is used to replace the upper positioning jig 300, and epoxy resin is injected or poured into the smart ring device 100, thereby encapsulating the outer ring 11, the one or more electronic components 20, the first electrode 30, the second electrode 40, the circuit board 70, and the curved battery assembly 80 together. After the epoxy resin cures, the isolation ring 60 or the inner ring 12 is formed, and a portion of the cured epoxy resin protrudes between the first electrode 30 and the second electrode 40 to form the isolation components 50.

It should be understood that, while the described examples utilize three jigs and the epoxy resin in the manufacturing procedure of the smart ring device 100, other types of jigs or moldable or potting material may be employed, which is not limited in the present disclosure.

In the smart ring device 100 provided by the present disclosure, the first electrode 30 and the second electrode 40 electrically contact the charging terminals 201 in the charging case 200 to achieve the charging function. The first electrode 30 and the second electrode 40 in contact with the skin may obtain various signals from the user, including at least one of electrodermal activity signals, body composition signals or temperature signals. The first electrode 30 and the second electrode 40 may integrate the charging function and the function of obtaining physiological signals of the user, making the smart ring device 100 simple in structure, saving the manufacturing cost of the smart ring device 100, while making the assembly relatively simple and ensuring the aesthetics of the smart ring device 100.

The foregoing descriptions are only some embodiments or implementations of the present disclosure, and are not intended to limit the present disclosure in any form. Any modification, equivalent replacement, improvement, etc., made within the content of the present disclosure shall be included in the protection scope of the present disclosure.

What is claimed is:

1. A smart ring device, comprising:
a housing, the housing comprising:
an outer housing, wherein an outer surface of the outer housing defines at least a portion of an outer surface of the housing;
an inner housing, wherein an inner surface of the inner housing defines at least a portion of an inner surface of the housing, and
an isolation layer provided between the outer housing and the inner housing, wherein the isolation layer comprises an electrically insulating material, and an accommodation space is defined at least in part between the isolation layer and at least one of the outer housing or the inner housing; and
one or more physiological sensors arranged within the accommodation space, wherein the one or more physiological sensors are configured to obtain one or more physiological measurements from a user when the smart ring device is worn by the user.

2. The smart ring device according to claim 1, wherein at least one of the outer housing or the inner housing comprises one or more electrically conductive materials.

3. The smart ring device according to claim 1, wherein the electrically insulating material of the isolation layer comprises at least one of a coating material or a potting material.

4. The smart ring device according to claim 1, wherein the electrically insulating material of the isolation layer comprises at least one of epoxy resin, polyurethane, polyethylene, or silicone.

5. The smart ring device according to claim 1, wherein the isolation layer comprises at least one of a coating layer or a potted layer comprising the electrically insulating material and being arranged on at least one of an inner surface of the outer housing or an outer surface of the inner housing.

6. The smart ring device according to claim 1, wherein the isolation layer is connected to a first one of the outer housing and the inner housing, and wherein the accommodation space is defined at least partially between the isolation layer and a second one of the outer housing and the inner housing.

7. The smart ring device according to claim 1, wherein a first electrode and a second electrode are provided along a circumferential direction of the housing and are electrically connected to at least one of the one or more physiological sensors.

8. The smart ring device according to claim 7, wherein the inner housing comprises a first portion comprising one or more first electronic conductive materials, a second portion comprising one or more second electronic conductive materials, and an isolation component arranged between the first portion and the second portion and comprising one or more electronic insulating materials, and wherein the first electrode comprises at least part of the first portion, and the second electrode comprises at least part of the second portion.

9. The smart ring device according to claim 7, wherein at least one aperture is provided on the inner surface of the inner housing, the first electrode and the second electrode protrude from the inner surface of the inner housing through the at least one aperture, and an electrically insulating layer is provided between the inner surface of the inner housing and at least one of the first electrode or the second electrode to electrically insulate the at least one of the first electrode or the second electrode.

10. The smart ring device according to claim 7, wherein each of the first electrode and the second electrode includes a first end and a second end opposite the first end along a circumferential direction of the housing, and the first electrode and the second electrode define at least one opening positioned in correspondence with at least one optical component disposed in the accommodation space, the at least one opening comprising at least one of:
a first opening located in an intermediate region circumferentially between the first end and the second end of the first electrode or the second electrode, or
a second opening collectively defined by the first electrode and the second electrode, the second opening including a first portion at the first end of the first electrode and a second portion at the first end of the second electrode.

11. The smart ring device according to claim 1, wherein the one or more physiological sensors comprise an optical sensor, the optical sensor comprising at least one light emitter and at least one light detector arranged along a circumferential direction of the housing.

12. The smart ring device according to claim 11, wherein the inner housing comprises an opaque material, and wherein at least one aperture is provided on the inner housing and positioned in correspondence with the at least one light emitter and the at least one light detector, and at least one light-transmitting lens is arranged within the at least one aperture and protrudes from the inner surface of the inner housing.

13. The smart ring device according to claim 11, wherein the at least one light detector comprises a first light detector and a second light detector, the first light detector and the second light detector being arranged on opposite sides of the at least one light emitter along the circumferential direction of the housing, and wherein the at least one light emitter is configured to emit light signals associated with at least two wavelengths.

14. The smart ring device according to claim 11, wherein a first electrode and a second electrode are provided along a circumferential direction of the housing and connected to at least one of the one or more physiological sensors, and the at least one light emitter is arranged between the first electrode and the second electrode along the circumferential direction of the housing.

15. The smart ring device according to claim 11, wherein at least one convex lens portion is arranged on the inner surface of the inner housing positioned in correspondence with at least part of the at least one light emitter and the at least one light detector, and wherein the at least one convex lens portion is configured to mate with at least one guide groove of a charging case when the smart ring device is received in the charging case for charging.

16. The smart ring device according to claim 1, wherein the one or more physiological sensors comprise at least one of an electrodermal activity sensor or a temperature sensor, wherein the electrodermal activity sensor is configured to obtain electrodermal activity signals of the user for emotion monitoring, and the temperature sensor is configured to obtain temperature signals of the user for body temperature monitoring.

17. The smart ring device according to claim 1, further comprising a circuit board and a curved battery assembly arranged within the accommodation space, wherein at least a portion of the circuit board and at least a portion of the curved battery assembly are arranged in different regions of the accommodation space and collectively define an annular or substantially annular arrangement along a circumferential direction of the housing.

18. The smart ring device according to claim 1, further comprising a rigid-flex circuit board arranged within the accommodation space, wherein the rigid-flex circuit board comprises at least two rigid circuit boards and at least one flexible circuit board connecting the at least two rigid circuit boards, and wherein at least one of the one or more physiological sensors is connected to the rigid-flex circuit board.

19. The smart ring device according to claim 1, wherein at least one of the outer housing or the inner housing comprises one or more metallic materials, and the isolation layer comprises one or more potting materials.

20. A smart ring device, comprising:
a housing comprising:
an outer ring, wherein an outer surface of the outer ring defines at least a portion of an outer surface of the housing;
an inner ring, wherein an inner surface of the inner ring defines at least a portion of an inner surface of the housing, and
at least one of a coating layer or a potted layer provided between the outer ring and the inner ring; and
one or more physiological sensors configured to obtain one or more physiological measurements from a user when the smart ring device is worn by the user, wherein the one or more physiological sensor is disposed within an accommodation space defined at least in part between at least one of the coating layer or the potted layer and at least one of the outer ring or the inner ring.
